(19) United States
(12) Patent Application Publication
(10) Pub. No.: US 2013/0150967 A1
(43) Pub. Date: Jun. 13, 2013

(54) Interbody Cage for Spine Fusion

(71) Applicants: Wei-Jen Shih, Kaohsiung City (TW); Yu-Ting Lyu, Kaohsiung City (TW); Wei-Te Chen, Kaohsiung City (TW); Yen-Nien Chen, Kaohsiung City (TW)

(72) Inventors: Wei-Jen Shih, Kaohsiung City (TW); Yu-Ting Lyu, Kaohsiung City (TW); Wei-Te Chen, Kaohsiung City (TW); Yen-Nien Chen, Kaohsiung City (TW)

(73) Assignee: Metal Industries Research & Development Centre, Kaohsiung City (TW)

(21) Appl. No.: 13/340,902

(22) Filed: Dec. 30, 2011

(30) Foreign Application Priority Data
Dec. 9, 2011 (TW) 100145601

(51) Int. Cl. A61F 2/44

(52) U.S. Cl. 623/17.11

(57) ABSTRACT

An interbody cage including a substrate and a plurality of through holes is disclosed. The substrate has a plurality of curve slits, with each of the curve slits having a plurality of sections, wherein a plurality deformable ribs is formed, with each deformable rib between any two curve slits and having a narrow part and a wide part. The plurality of through holes is formed in the wide part of the deformable rib. In use, any adjacent two of the deformable ribs are capable of being bent to depart from each other to define a filling room.

### BACKGROUND OF THE INVENTION

1. Field of the Invention

The present invention relates to an interbody cage and, more particularly, to an interbody cage for spine fusion, which is capable of enhancing the growing speed and stability of bones.

2. Description of the Related Art

Recently, cases of spinal diseases have occurred often since the numbers of office workers and the elders are increasing and the spinal diseases are common in these two groups. Therefore, how to take care of patients of spinal diseases has become more and more important.

Generally, medical attention of a spinal patient after an operation is extremely important for the patient's recovery, and the patient usually has to put on a spinal brace for a long time so that the spinal brace may provide an external support for the spine of the patient and prevent the recovering spine from injury again.

Nowadays, the way to improve the patient's recovery is to implant an interbody cage in a position adjacent to a bone defect in his spine, with filler such as Calcium Phosphate, bone graft, and auto-genous graft in the interbody cage. However, the filler inside the interbody cage may easily flow away due to intrusion of soft tissues, which usually grow faster than bone tissues, in the interbody cage or circulatory system if the meshes of the interbody cage are not well designed, and thus this implant made of the interbody cage and filler will not achieve a desirable performance. Referring to FIG. 1, a conventional interbody cage 9 disclosed by a

Taiwan patent with a patent number of M333884 is shown. The interbody cage 9 has a head 91 on the front side thereof, a room 92 formed behind the head 91, a plurality of holes 93 arranged in lateral walls of the interbody cage 9 and communicating with the room 92, a base 94 on the rear side of the interbody cage 9, and a fixing seat 95 for the base 94 to firmly mount on. The room 92 is arranged for the filler to insert, and the filler inside the interbody cage 9 is accessible to the bone cells of a spine through the holes 93 for spine fusion.

However, there should be a plurality of screws 96 coupling with the is fixing seat 95 to firmly position the interbody cage 9 in a position adjacent to a bone defect, and this conventional interbody cage 9 is suitable to be positioned between two vertebral columns only. Moreover, the complexity, difficulty in made and manufacture cost of this conventional interbody cage 9 are high since the interbody cage 9 can only be shaped by 3D laser carving and spark-discharge forming. Additionally, in medical operation, this complex interbody cage 9 also leads to a long time period in assembly of the screws 96 and the fixing seat 95, and thus the possibility of bacterial infection in medical operation can be largely raised.

Furthermore, the filler loss problem due to soft tissues' intrusion and circulatory system may easily occur in this interbody cage 9 since the room 92 and holes 93 are not particularly designed to block the intruded soft tissues and the filler inside the room 92. Accordingly, the conventional interbody cage 9 cannot effectively enhance the growing speed of a defective bone but only serves as a support, and thus the interbody cage 9 cannot help a lot in recovery of the bone.

Therefore, a titanium-based interbody cage for enhancing the growing speed and stability of defective bones and suitable for use in any part of the spine is needed to solve the above problems.

### SUMMARY OF THE INVENTION

It is therefore the primary objective of this invention to provide an interbody cage with a simple and integral structure without any joint between any two separate members, so as to lower the difficulty and cost in manufacture.

Another objective of this invention is providing an interbody cage to prevent the soft tissues from intrusion and to surely hold the filler so as to avoid the filler flowing away.

Still another objective of this invention is providing an interbody cage suitable to be used in any position of a patient with spinal disease for enhancing the growing of the bone tissues via the filler inside this interbody cage.

The invention discloses an interbody cage including a substrate and a plurality of through holes. The substrate has a plurality of curve slits, with each of the curve slits having a plurality of sections, wherein a plurality deformable ribs is formed, with each deformable rib between any two curve slits and having a narrow part and a wide part. The plurality of through holes is formed in the wide part of the deformable rib. Any adjacent two of the deformable ribs are capable of being bent to depart from each other to define a filling room.

The invention further discloses that a biodegradable macromolecular film is formed on one side of the substrate to totally or partially cover the said side.

The invention further discloses that a thickness of the substrate is 20-200 micrometers, and the diameters of the through holes are in a range between 1-3 mm.

The invention further discloses the substrate has two lateral bars on two opposite ends thereof, each curve slit extends from one of the lateral bars to the other one of the lateral bars and is spaced from two opposite edges of the substrate by the lateral bars, and each deformable rib has a first end and a second end respectively connecting with the two lateral bars.

The invention further discloses that each section of any one of the curve slits has a first part and a second part in arc shapes, the first parts and the second parts of the sections of any one of the curve slits are arranged in a stagger way, curvature centers of the first parts are on one side of the curve slit, and curvature centers of the second parts are on the other side of the curve slit.

The invention further discloses that each narrow part of any one of the deformable ribs is formed between a first part and a second part of adjacent two of the curve slits defining the deformable rib with centers of the said first and second parts being bent towards each other, and each wide part of any one of the deformable ribs is formed between a first part and a second part of adjacent two of the curve slits defining the deformable rib with centers of the said first and second parts being bent away from each other.

The invention further discloses that the wide parts are in a circular shape and the through holes in the wide parts are in a circular shape concentric to the circular shape of the wide parts.

The invention further discloses that the wide parts are in an eye-like shape and the through holes in the wide parts are also in an eye-like shape.

The invention further discloses that the wide parts are in a circular shape, a part of the wide parts has the through holes in a circular shape, the other part of the wide parts has the through holes in a bar shape, and the wide parts having the circular through holes and wide parts having the bar-shaped through holes are arranged by stagger.

The invention further discloses that each first part is in an S shape and each second part is in an arc shape.

The invention further discloses that the narrow part of one of the deformable ribs is in an S shape and the wide part of the deformable rib is in a circle shape, with a circular through hole formed in the wide part and concentric to the circular shape of the wide part.

The invention further discloses that the wide parts are in a rhombus shape.

The invention further discloses that the through holes in the wide parts are formed in an ellipse shape.

The invention further discloses that the wide parts are in a rectangle shape.

The invention further discloses that the through holes in the wide parts are formed in an oval shape.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 shows a perspective view of a conventional interbody cage.

FIG. 2 shows a perspective view of an interbody cage according to a preferable embodiment of the invention in a 2D flat piece form.

FIG. 3 shows a perspective view of the interbody cage in a 3D cage form.

FIGS. 4-8 show top views of various interbody cages according to the preferable embodiment of the invention.

FIG. 9 shows a sketch diagram of the interbody cage in use.

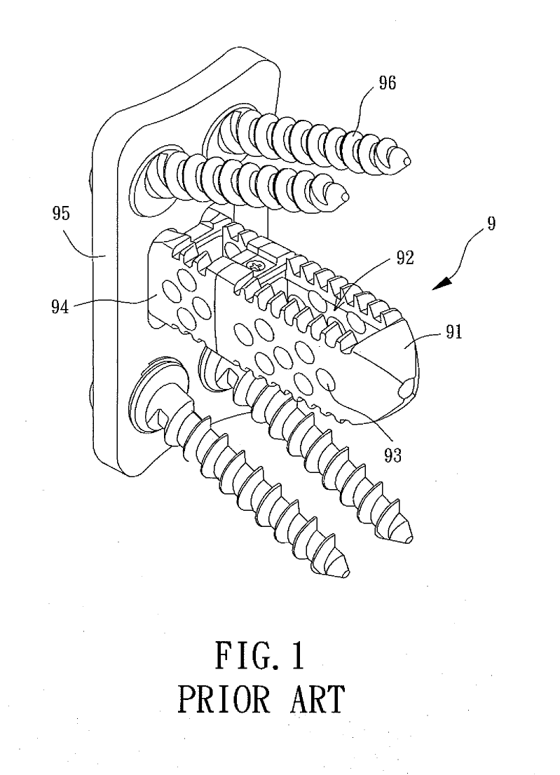

In the various figures of the drawings, the same numerals designate the same or similar parts. Furthermore, when the term “first,” “second,” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

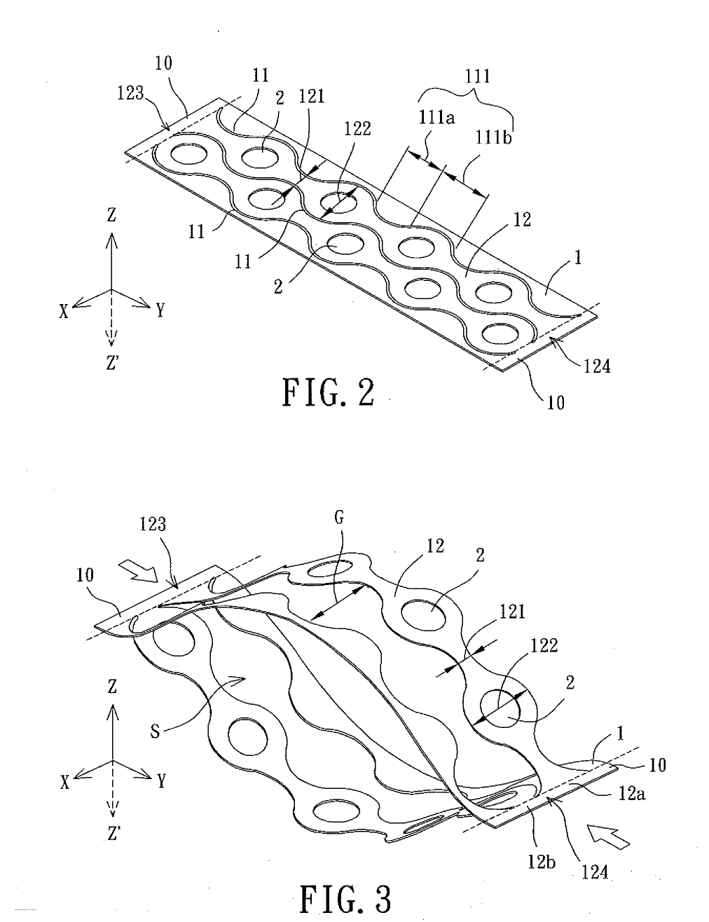

FIG. 2 shows an interbody cage according to a preferable embodiment of the invention, which includes a substrate 1 and a plurality of through holes 2. The plurality of through holes 2 penetrates the substrate 1 from one side of the substrate 1 to an opposite side thereof so as to communicate these two opposite sides of the substrate 1. The substrate 1 is preferably made of pure titanium or titanium alloy with a thickness of 20-200 micrometers between the two opposite sides, so that the substrate 1 is easy to be deformed into a specific shape during a medical operation.

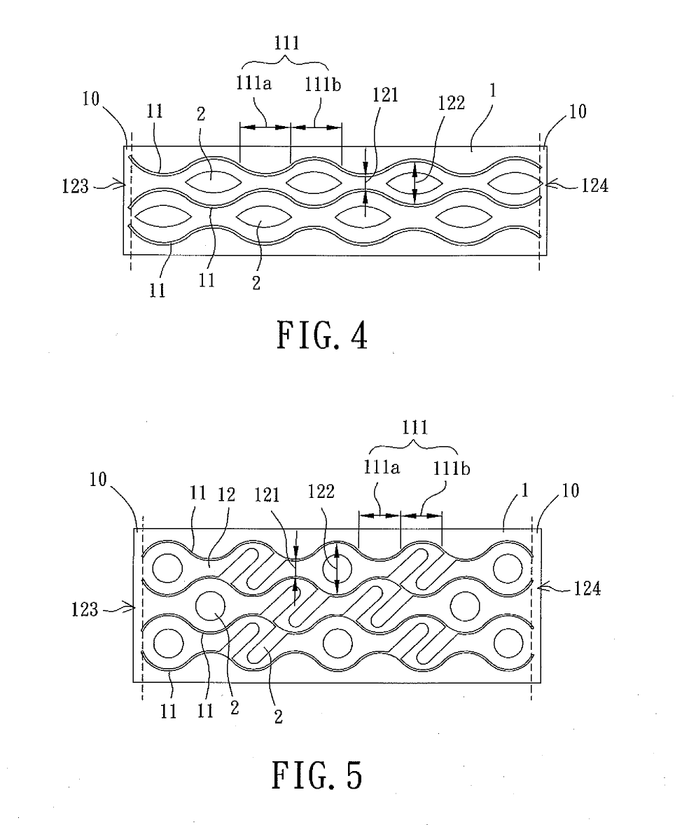

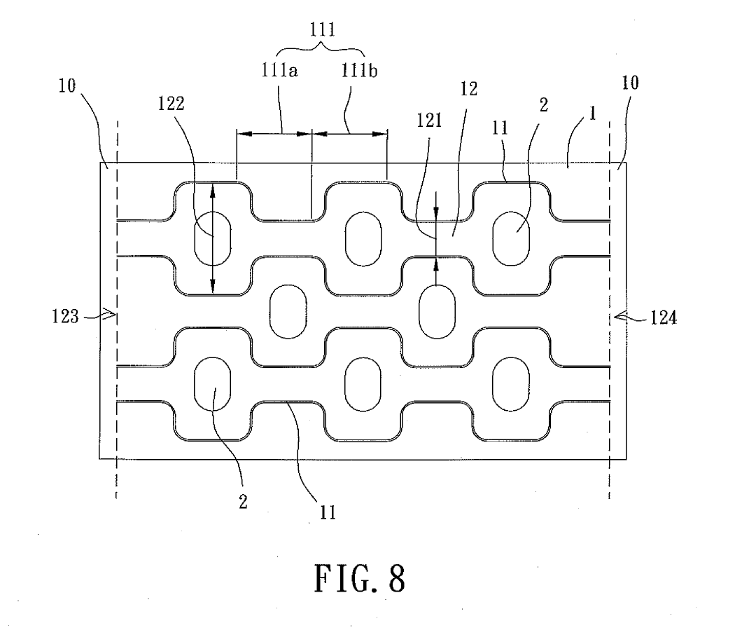

The substrate 1 has two lateral bars 10 on two opposite ends of the substrate 1 and a plurality of curve slits 11 extending between the two lateral bars 10. Each of the curve slits 11 has a plurality of sections 111, wherein routes of the curve slits 11 in the sections 111 may be the same or different so as to form the sections 111 with repeated or random shapes. Specifically, each slit 11 of the substrate 1 in the present example shown by FIGS. 2 and 3 has continuous and repeated sections 111. However, routes of the curve slits 11 are not limited, while various examples of the curve slit 11 are shown in FIGS. 4-8 and illustrated later.

Referring to FIG. 2 again, each section 111 of any one of the curve slits 11 has a first part 111a and a second part 111b, while the first parts 111a and the second parts 111b of the sections 111 of the same curve slit 11 are arranged in a stagger way. Particularly, it is preferable that both of the first part 111a and the second part 111b are in arc shapes, curvature centers of the first parts 111a are on one side of the curve slit 11, and curvature centers of the second parts 111b are on the other side of the curve slit 11, so that each curve slit 11 is in a S-shape. Preferably, each curve slit 11 extends from one of the lateral bars 10 to the other one of the lateral bars 10 and is spaced from two opposite edges of the substrate 1 by the lateral bars 10. For example, as those shown in FIG. 2, each curve slit 11 extends from one of the lateral bars 10 close to a left edge to the other lateral bar 10 close to a right edge in a Y direction.

Referring to FIGS. 2 and 3, there is a deformable rib 12 between any two curve slits 11, so that the deformable rib 12 is bent while two external forces oppositely press the substrate 1 on the two lateral bars 10 as the arrows shown in FIG. 3. Therefore, the deformable ribs 12 can be bent, with any two adjacent ribs 12 being bent to depart from each other, and thus the substrate 1 is changed from a 2D flat piece into an integral 3D cage. In this embodiment, each deformable rib 12 has a plurality of narrow part 121 and a plurality of wide part 122. Each narrow part 121 of a deformable rib 12 is formed between a first part 111a and a second part 111b of two adjacent curve slits 11 defining the deformable rib 12, and centers of the said first and second parts 111a, 111b are bent towards each other. Contrarily, although each wide part 122 of the deformable rib 12 is also formed between a first part 111a and a second part 111b of two adjacent curve slits 11 defining the deformable rib 12, centers of the said first and second parts 111a, 111b are bent away from each other. In correspondence with the arrangement of the first parts 111a and second parts 111b of the curve slits 11, the narrow parts 121 and wide parts 122 may also be arranged in a stagger way.

Moreover, a first end 123 and a second end 124 of the deformable rib 12 are defined, with the first and second ends 123, 124 respectively connect with the two lateral bars 10, so that the first and second ends 123, 124 can be bent relative to the said lateral bars 10 of the deformable rib 12 while the external pressure enforces the two lateral bars 10 to get close, so as to form the 3D cage capable of receiving the filler.

The plurality of through holes 2 is formed in the wide parts 122 of the deformable rid 12, and may connects with the curve slit 11 if necessary. In the example shown in FIGS. 2 and 3, each one of the through holes 2 is in the shape of a circle and arranged in a respect one of the wide parts 122, with the curvature centers of the first part 111a and second part 111b defining any one of the wide parts 122 and the center of the through hole 2 in this wide part 122 being the same. In this example, the diameters of the through holes 2 are in a range between 0.5-3.5 mm, preferably in a range between 1-3 mm, more preferably of 2 mm, so as to lighten the substrate 1, improve the flexibility of the substrate 1, and lower the interference due to the 3D cage on X-ray films.

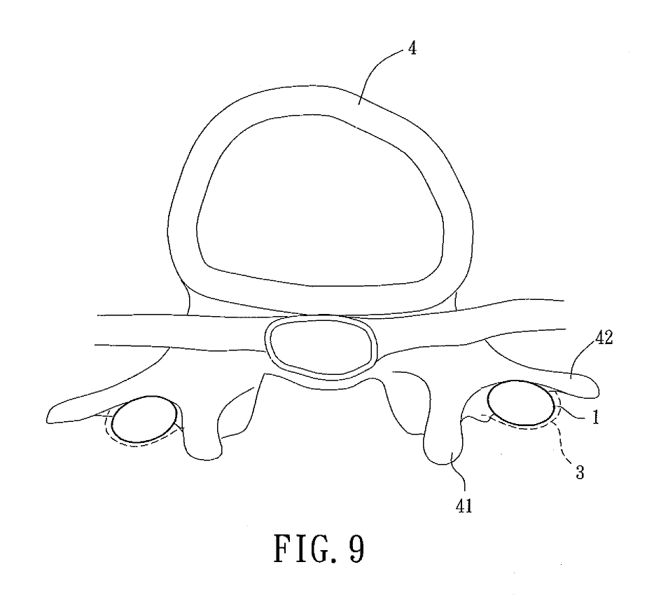

Referring to FIG. 9, a biodegradable macromolecular film 3 can be formed on one side of the substrate 1, which may totally or partially cover the said side. Preferably, the biodegradable macromolecular film 3 covers one side of the substrate 1 only, with the uncovered side of the substrate 1 facing the bone defect for the filler inside the 3D cage to enhance the growing of the bone tissues and the covered side of the substrate 1 capable of preventing the intrusion of the soft tissues. The biodegradable macromolecular film 3 may made of collagen, chitosan, gelatin or hyaluronan.

The interbody cage of the present invention can be made by the following method: selecting a titanium film with 50 micrometers as the substrate 1; forming the plurality of curve slits 11 and through holes 2 with the previous discussed structures by 2D laser carving; and soaking the carved substrate 1 into 37% hydrochloric acid for 30 minutes so as to lower the roughness of the surfaces of the substrate 1 to a degree smaller than 1.5 micrometers.

When the interbody cage of this invention is used, the substrate 1 has to be transformed from the 2D flat piece into the integral 3D cage. Referring to FIG. 3, the lateral bars 10 are moved toward each other by external forces in the directions parallel to the Y direction and shown by the arrows, so that any two adjacent deformable ribs 12 labeled as “12a” and “12b” respectively, which is originally aligned side by side to form the 2D flat piece, can be departed from each other to form the 3D cage. Specifically, the first and second ends 123, 124 of the deformable rib 12a serve as hinges for the deformable rib 12a to bend in a Z direction perpendicular to the Y direction, while the first and second ends 123, 124 of the deformable rib 12b serve as hinges for the deformable rib 12b to bend in a Z′ direction opposite to the Z direction. Therefore, the oppositely bent deformable ribs 12a, 12b can define a filling room “S” for receiving the filler. It should be noticed that a number of the deformable ribs 12 is changeable to adjust the length of the interbody cage upon need. It is preferable that the length of the interbody cage is that of 2-3 vertebral columns of the patient.

Particularly, a gap “G” is formed between two adjacent deformable ribs 12a or two adjacent deformable ribs 12b, which are on the same side of the filling room “S,” and is in a shape identical to that of a deformable rib 12b or a deformable rib 12a since the filling room “S” is formed by the oppositely bent deformable ribs 12a, 12b. Once the filler is filled into the filling room “S” in use, the filler is accessible to the bone tissues via the gap “G.” As a result, it is preferable that the width of the wide part 122 is smaller than that of the filler, with the width of the wide part 122 being 1.5-4.5 mm and more preferably being 2-4 mm, so that the filler is firmly held inside the filling room “S” and cannot fall out of the interbody cage through the gap “G;” so as to effectively enhance the recovery of the bone defect.

Referring to FIGS. 4-8, the interbody cages of the preferable embodiment of this invention with various curve slits 11 and through holes 2 having different shapes are shown. It should be noticed that the arrangements of the curve slits 11 and through holes 2 can be adjusted to improve the strength and smoothness of the bent deformable rids 12, and thus the deformable rids 12 will not be easily broken or hurt the bone tissues nearby via sharp corners thereof.

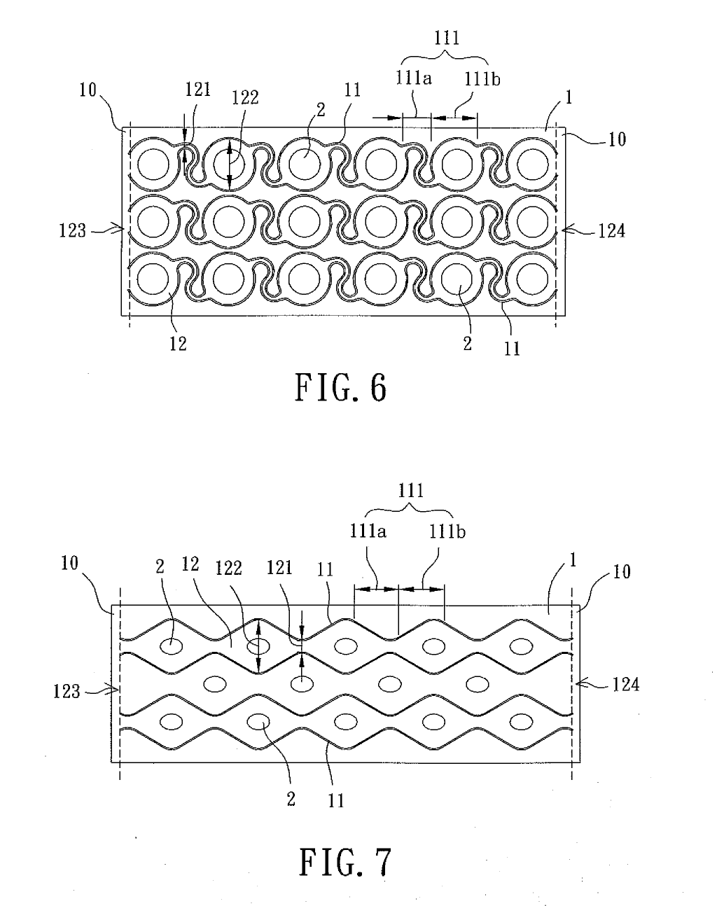

Referring to FIG. 4, the first part 111a and second part 111b defining any one of the wide parts 122 are arranged to form the wide part 122 into an eye-like shape, while the through holes 2 in the wide parts 122 are also correspondingly formed in an eye-like shape. Referring to FIG. 5, the first part 111a and second part 111b defining any one of the wide parts 122 are arranged to form the wide part 122 into a circle, wherein a part of the wide parts 122 has the through holes 2 in a circular shape and the other part of the wide parts 122 has the through holes 2 in a bar shape. Preferably, the wide parts 122 having the circular through holes 2 and wide parts 122 having the bar-shaped through holes 2, which are the wide parts 122 of the same deformable rib 12, are arranged by stagger. Referring to FIG. 6, the first parts 111a are in an S shape and the second parts 111b are in an arc shape, so that the narrow part 121 of a deformable rib 12 is in an S shape and the wide part 122 is in a circular shape with a circular through hole 2 formed in the wide part 122. Referring to FIG. 7, the first part 111a and second part 111b defining any one of the wide parts 122 are arranged to form the wide part 122 into a rhombus shape, while the through holes 2 in the wide parts 122 are formed in an ellipse shape. Referring to FIG. 7, the first part 111a and second part 111b defining any one of the wide parts 122 are arranged to form the wide part 122 into a rectangle shape, while the through holes 2 in the wide parts 122 are formed in an oval shape. The above illustrated examples are shown for illustration but not limit the practice of this invention.

Referring to FIG. 9, when the interbody cage of the present invention is used in a medical operation, the filler is initially filled into the filling room “S” formed by the bent deformable ribs 12b of the pressed substrate 1, and then the interbody cage with the filler may be disposed in a suitable position near the bone defect directly or by thread without any nail for fixing the interbody cage. The said suitable position is preferably between the cervical spine 41 and the transverse spine 42 of the spine 4 of the patient.

In sum, the interbody cage of the present invention is characterized in the following features. First, the interbody cage is easy for storage before use since the substrate 1 is a 2D flat piece merely. Second, the interbody cage is convenient for use since the deformable ribs 12 can be spread out easily by the opposite external forces on the lateral bars 10 to form the 3D cage. Third, the interbody cage is strong since the substrate 1 is integrally formed without any joint between any two separate members, so that the structure of the interbody cage will not be loosened or broken from the joint. Fourth, the routes of the curve slits 11 can be well designed to avoid weak points of the deformable ribs 12 and hurt to the bone. Fifth, the interbody cage has a reduced weight, improved flexibility and lower interference on X-ray films since the through holes 2 are formed in the deformable rids 12. Thereby, the present interbody cage not only can be manufactured easily, but can efficiently hold the filler as well as efficiently prevent the soft tissues from intrusion via the applied biodegradable macromolecular film 3. Accordingly, the present interbody cage can provide a sufficient strength for support as well as improve the growing of the bone tissues.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. An interbody cage, comprising:
a substrate having a plurality of curve slits, with each of the curve slits having a plurality of sections, wherein a plurality of deformable ribs is formed, with each deformable rib between any adjacent two of the curve slits and having a narrow part and a wide part; and
a plurality of through holes formed in the wide part of the deformable rib;
wherein any adjacent two of the deformable ribs are capable of being bent to depart from each other to define a filling room.

2. The interbody cage as claimed in claim 1, wherein a biodegradable macromolecular film is formed on one side of the substrate to totally or partially cover the said side.

3. The interbody cage as claimed in claim 1, wherein a thickness of the substrate is 20-200 micrometers, and the diameters of the through holes are in a range between 1-3 mm.

4. The interbody cage as claimed in claim 1, wherein the substrate has two lateral bars on two opposite ends thereof, each curve slit extends from one of the lateral bars to the other one of the lateral bars and is spaced from two opposite edges of the substrate by the lateral bars, and each deformable rib has a first end and a second end respectively connecting with the two lateral bars.

5. The interbody cage as claimed in claim 1, wherein each section of any one of the curve slits has a first part and a second part in arc shapes, the first parts and the second parts of the sections of any one of the curve slits are arranged in a stagger way, curvature centers of the first parts are on one side of the curve slit, and curvature centers of the second parts are on the other side of the curve slit.

6. The interbody cage as claimed in claim 5, wherein each narrow part of any one of the deformable ribs is formed between a first part and a second part of adjacent two of the curve slits defining the deformable rib with centers of the said first and second parts being bent towards each other, and each wide part of any one of the deformable ribs is formed between a first part and a second part of adjacent two of the curve slits defining the deformable rib with centers of the said first and second parts being bent away from each other.

7. The interbody cage as claimed in claim 6, wherein the wide parts are in a circular shape and the through holes in the wide parts are in a circular shape concentric to the circular shape of the wide parts.

8. The interbody cage as claimed in claim 6, wherein the wide parts are in an eye-like shape and the through holes in the wide parts are also in an eye-like shape.

9. The interbody cage as claimed in claim 6, wherein the wide parts are in a circular shape, a part of the wide parts has the through holes in a circular shape, the other part of the wide parts has the through holes in a bar shape, and the wide parts having the circular through holes and wide parts having the bar-shaped through holes are arranged by stagger.

10. The interbody cage as claimed in claim 1, wherein each first part is in an S shape and each second part is in an arc shape.

11. The interbody cage as claimed in claim 10, wherein the narrow part of one of the deformable ribs is in an S shape and the wide part of the deformable rib is in a circle shape, with a circular through hole formed in the wide part and concentric to the circular shape of the wide part.

12. The interbody cage as claimed in claim 1, wherein the wide parts are in a rhombus shape.

13. The interbody cage as claimed in claim 12, wherein the through holes in the wide parts are formed in an ellipse shape.

14. The interbody cage as claimed in claim 1, wherein the wide parts are in a rectangle shape.

15. The interbody cage as claimed in claim 14, wherein the through holes in the wide parts are formed in an oval shape.
